(19) United States
(12) Patent Application Publication
(10) Pub. No.: US 2022/0305167 A1
(43) Pub. Date: Sep. 29, 2022

(54) Atomizer with receptacle at low pressure

(71) Applicant: Gang Wei, Shenzhen (CN)

(72) Inventor: Gang Wei, Shenzhen (CN)

(21) Appl. No.: 17/806,946

(22) Filed: Jun. 15, 2022

(51) Int. Cl. A61L 9/14

(52) U.S. Cl. CPC A61L 9/14; A61L 2209/134

(57) ABSTRACT

An essential oil atomizer includes an essential oil receptacle in a housing; first and second fluid connectors connected to the essential oil receptacle; a water reservoir on a bottom of the housing; first and second water lines connected to the water reservoir; a first flexible tube interconnected to the first water line and the first fluid connector; a second flexible tube interconnected to the second water line and the second fluid connector; a bracket on the water reservoir; a peristaltic pump on the bracket; two openings on one side of the peristaltic pump; and an electric motor on one surface of the peristaltic pump. The first flexible tube passes through one opening, inside of the peristaltic pump and the other opening. The peristaltic pump includes a rotatable disc operatively connected to the electric motor, three shafts secured to rotatable disc, and three bearings put on the shafts respectively.

### FIELD OF THE INVENTION

The invention relates to essential oil atomizers and more particularly to such an essential oil atomizer having an essential oil receptacle having an internal pressure less than the atmospheric pressure.

### BACKGROUND OF THE INVENTION

Essential oils are used to improve the surrounding environment or to perform medical treatment. There are conventional essential oil atomizers which are used to atomize the essential oil for diffusing same into the environment.

However, the conventional essential oil atomizers have the following disadvantages: prior to activation, an essential oil receptacle is full of air. Water is pumped into the essential oil receptacle through a second flexible tube by a peristaltic pump. Excess fluid may flow to a first flexible tube. Unfortunately, fluid may leak out of an atomization member because air in the essential oil receptacle causes internal pressure of the essential oil receptacle to be about equal to the atmospheric pressure.

Thus, the need for improvement still exists.

### SUMMARY OF THE INVENTION

The invention has been made in an effort to solve the problems of the conventional art including leakage by providing an essential oil atomizer having an essential oil receptacle having an internal pressure less than the atmospheric pressure with novel and nonobvious characteristics.

To achieve above and other objects of the invention, the invention provides an essential oil atomizer comprising an essential oil atomizer, comprising a housing; an essential oil receptacle disposed in the housing; first and second fluid connectors connected to the essential oil receptacle; a water reservoir disposed on a bottom of the housing; first and second water lines connected to the water reservoir; a first flexible tube interconnected to the first water line and the first fluid connector; a second flexible tube interconnected to the second water line and the second fluid connector; a bracket disposed on the water reservoir; a peristaltic pump disposed on the bracket; two openings disposed on one side of the peristaltic pump; and an electric motor disposed on one surface of the peristaltic pump; wherein the first flexible tube passes through one opening, inside of the peristaltic pump, and the other opening; and the peristaltic pump includes a rotatable disc operatively connected to the electric motor, three equally spaced shafts secured to rotatable disc, and three bearings put on the shafts respectively.

The essential oil atomizer of the invention has the following advantageous effects in comparison with the prior art: The first flexible tube is taken as an inlet tube rather than the second flexible tube taken as an inlet tube in the conventional art. Further, the peristaltic pump counterclockwise rotates rather than clockwise rotation of the peristaltic pump in the conventional art. The problem of leakage of the conventional art is solved. The counterclockwise rotated peristaltic pump may compress the second flexible tube to flow fluid in the essential oil receptacle to the water reservoir through the second flexible tube and the second water line. The atmospheric pressure is greater than internal pressure of the essential oil receptacle. The atomized particles do not leak out of the atomization member due to the greater atmospheric pressure. Air flows from the second flexible tube to the atomization member and water flows from the first flexible tube to the atomization member. Water does not leak out of the atomization member. The water flow will slow if the level of water in the water reservoir is low. This is because air enters the atomization member. It may take a longer time to draw liquid out of the essential oil receptacle. Air in the second flexible tube and the second water line is almost completely drawn out. The liquid may be quickly drawn out if there is liquid in the atomization member.

Moreover, the second flexible tube is taken as an inlet tube and subsequently the first flexible tube is taken as the inlet tube rather than the second flexible tube taken as an inlet tube in the conventional art. Further, the peristaltic pump clockwise rotates and then counterclockwise rotates rather than the clockwise rotation of the peristaltic pump in the conventional art. Thus, the problem of leakage of the conventional art is solved. The second flexible tube is taken as the inlet tube when the peristaltic pump clockwise rotates. Water in the water reservoir flows to the essential oil receptacle through the second water line and the second flexible tube. Next, air is drawn out of the second water line, the second flexible tube and the essential oil receptacle. At this time, liquid is not fully drawn out such that liquid does not leak out of the atomization member. Next, the peristaltic pump counterclockwise rotates and the first flexible tube is taken as the inlet tube. As a result, the liquid pumping efficiency is greatly increased because air has been drawn out of the second water line, the second flexible tube and the essential oil receptacle. Therefore, the problem of leakage of the atomization member is solved.

The above and other objects, features and advantages of the invention will become apparent from the following detailed description taken with the accompanying drawings.

### BRIEF DESCRIPTION OF THE DRAWINGS

FIG. 1 is a front, top and right side perspective view of an essential oil atomizer of the invention;

FIG. 2 is a rear, bottom and left side perspective view of the essential oil atomizer;

FIG. 3 is a front, top and left side perspective view of the essential oil atomizer with the housing removed;

FIG. 4 is a front, top and right side perspective view of the essential oil atomizer with the housing removed; and

FIG. 5 is a longitudinal sectional view of the peristaltic pump.

### DETAILED DESCRIPTION OF THE INVENTION

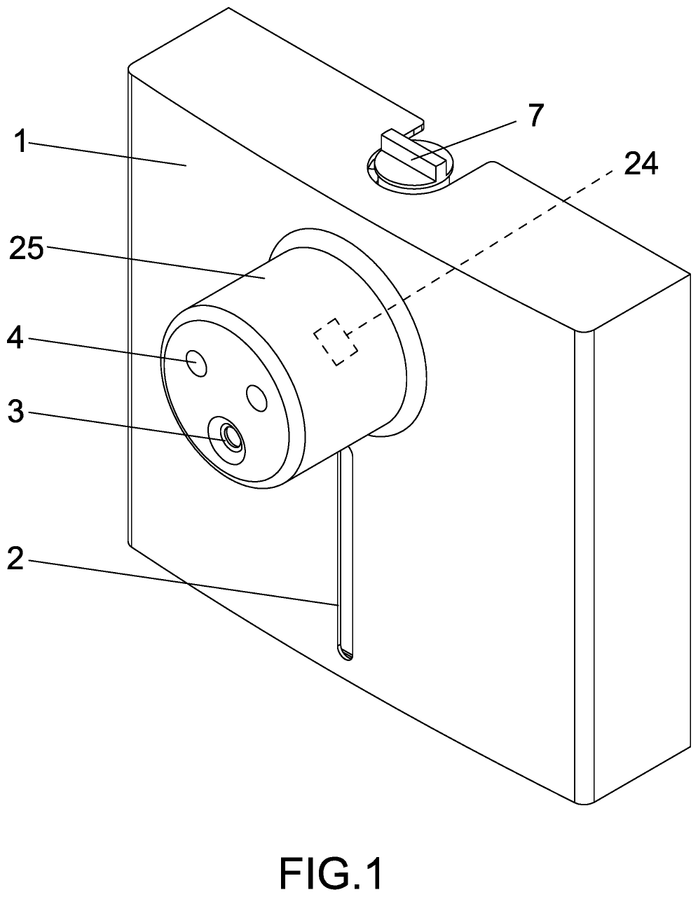

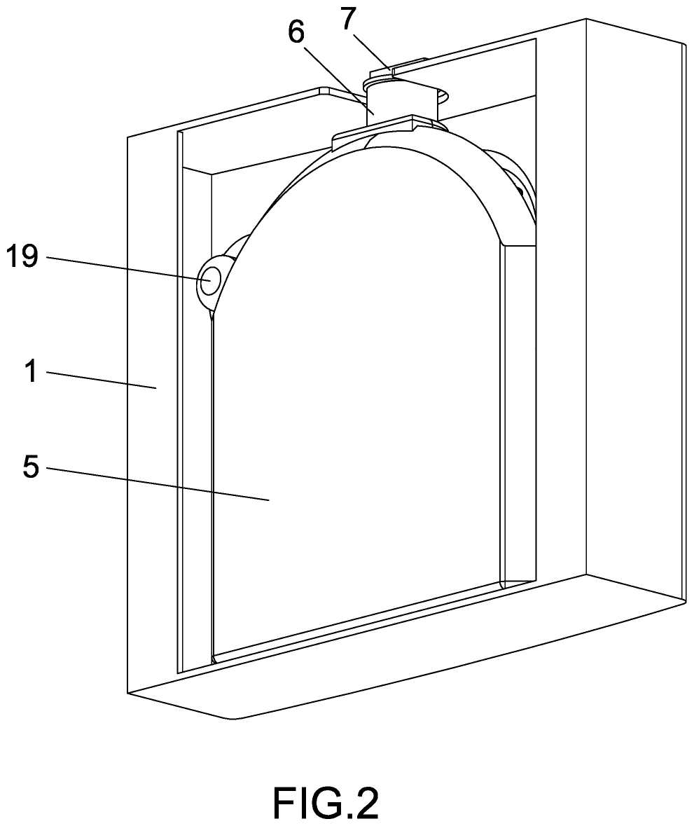

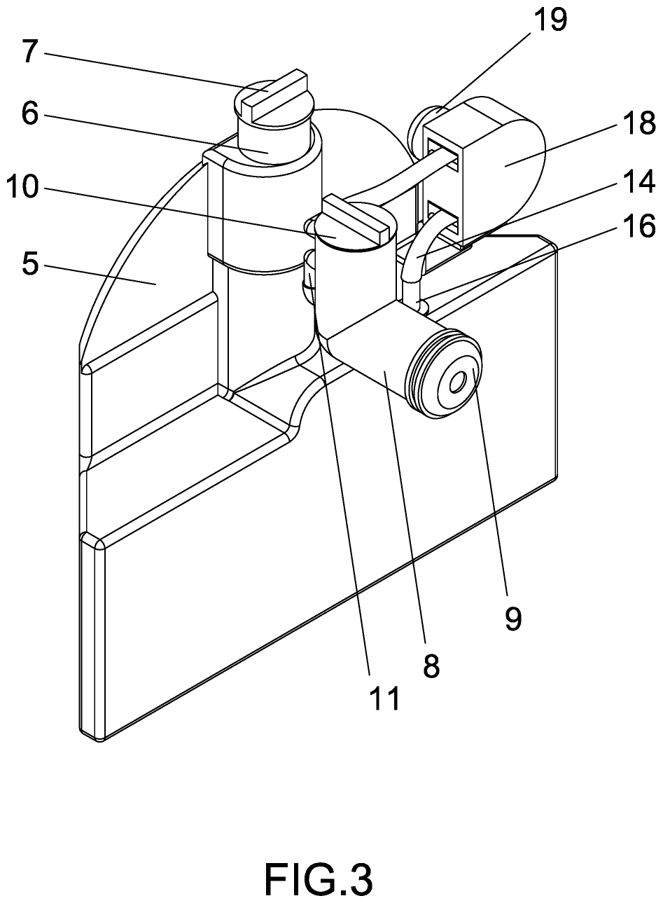

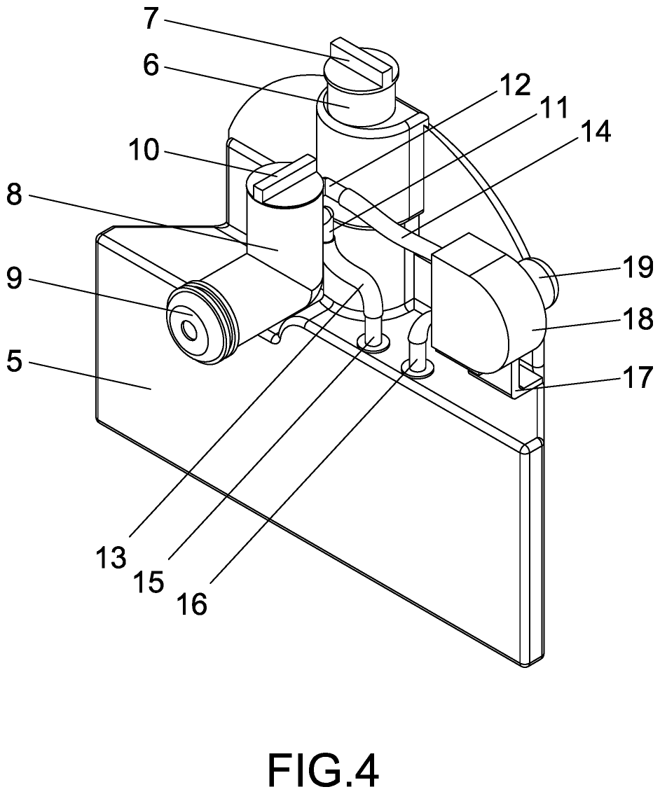

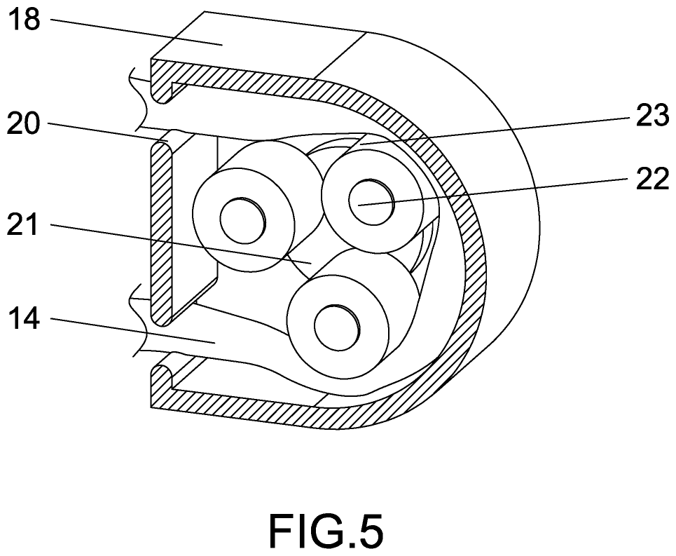

Referring to FIGS. 1 to 5, an essential oil atomizer in accordance with the invention comprises the following components as discussed in detail below.

An L-shaped essential oil receptacle 8 is provided in a housing 1. First and second fluid connectors 11 and 12 are connected to the essential oil receptacle 8. A water reservoir 5 is provided on a bottom of the housing 1. First and second water lines 15 and 16 are connected to the water reservoir 5. A first flexible tube 13 is interconnected to the first water line 15 and the first fluid connector 11 and a second flexible tube 14 is interconnected to the second water line 16 and the second fluid connector 12. A bracket 17 is provided on the water reservoir 5. A peristaltic pump 18 is provided on the bracket 17. Two openings 20 are provided on one side of the peristaltic pump 18. The first flexible tube 14 passes through one opening 20, inside of the peristaltic pump 18 and the other opening 20. An electric motor 19 is provided on one surface of the peristaltic pump 18. Within the peristaltic pump 18, there are provided a rotatable disc 21 operatively connected to a motor shaft of the electric motor 19, three equally spaced shafts 22 secured to rotatable disc 21, and three bearings 23 are put on the shafts 22 respectively.

Embodiment 1. The first fluid connector 11 is below the second fluid connector 12. Both the first and second water lines 15 and 16 are inserted into the water reservoir 5. Fluid in the essential oil receptacle 8 flows through the first fluid connector 11, the first flexible tube 13, the first water line 15, the second fluid connector 12, and the second flexible tube 14 into the first water line 15. The motor shaft of the electric motor 19 is secured to a center of the rotatable disc 21. The second flexible tube 14 passes through a space between an inner surface of the peristaltic pump 18 and the bearings 23. A slit 2 is provided on the housing 1 and aligned with the water reservoir 5. A user can see level of water in the water reservoir 5. A cylindrical member 25 projects out of the housing 1. A nozzle 3 is provided at an end of the cylindrical member. An inlet 6 is provided on a top of the water reservoir 5 and a sealing cap 7 is provided on the inlet 6. The inlet 6 and the sealing cap 7 are threadedly secured together. Water can be poured into the water reservoir 5 through the inlet 6.

An atomization member 9 is provided at an end of the essential oil receptacle 8. A sealing cap 10 is provided at the other end of the essential oil receptacle 8 and threadedly secured thereto. The atomization member 9 is aligned with the nozzle 3. Two buttons 4 are provided on the end of the cylindrical member adjacent to the nozzle 3. A controller 24 is provided in the cylindrical member and electrically connected to the buttons 4, the atomization member 9 and the electric motor 19. Further, the controller is electrically connected to mains electricity.

The first flexible tube 13 is taken as an inlet tube rather than the second flexible tube 14 taken as an inlet tube in the conventional art. Further, the peristaltic pump 18 counterclockwise rotates rather than clockwise rotation of the peristaltic pump 18 in the conventional art. Thus, the problem of leakage of the conventional art is solved. The counterclockwise rotated peristaltic pump 18 may compress the second flexible tube 14 to flow fluid in the essential oil receptacle 8 to the water reservoir 5 through the second flexible tube 14 and the second water line 16. The atmospheric pressure is greater than internal pressure of the essential oil receptacle 8. The atomized particles do not leak out of the atomization member 9 due to the greater atmospheric pressure. Air flows from the second flexible tube 14 to the atomization member 9 and water flows from the first flexible tube 13 to the atomization member 9. Water does not leak out of the atomization member 9. The water flow will slow if the level of water in the water reservoir 5 is low. This is because air enters the atomization member 9. It may take a longer time to draw liquid out of the essential oil receptacle 8. Air in the second flexible tube 14 and the second water line 16 is almost completely drawn out. The liquid may be quickly drawn out if there is liquid in the atomization member 9.

Embodiment 2. The second flexible tube 14 is taken as an inlet tube and then the first flexible tube 13 taken as an inlet tube. Further, the peristaltic pump 18 clockwise rotates and then counterclockwise rotates. Thus, the problem of leakage of the conventional art is solved. The second flexible tube 14 is taken as the inlet tube when the peristaltic pump 18 clockwise rotates. Water in the water reservoir 5 flows to the essential oil receptacle 8 through the second water line 16 and the second flexible tube 14. Next, air is drawn out of the second water line 16, the second flexible tube 14 and the essential oil receptacle 8. At this time, liquid is not fully drawn out such that liquid does not leak out of the atomization member 9. Next, the peristaltic pump 18 counterclockwise rotates and the first flexible tube 13 is taken as the inlet tube. As a result, the liquid pumping efficiency is greatly increased because air has been drawn out of the second water line 16, the second flexible tube 14 and the essential oil receptacle 8. Therefore, the problem of leakage of the atomization member 9 is solved.

Operation principle is described in detail below. Two method of filling the essential oil receptacle 8 with essential oil are used for preventing liquid from leaking out of the atomization member 9.

First method involves the steps of electrically connecting the controller 24 to the mains electricity; putting the essential oil atomizer on a table or mounting the essential oil atomizer on a wall; filling the water reservoir 5 with water through the inlet 6; pressing the button 4 to activating the essential oil atomizer; the control 24 activating the electric motor 19 to cause the electric motor 19 to clockwise rotate; clockwise rotating the rotatable disc 21 so that the bearings 23 on the shafts 22 clockwise rotate; the bearings 23 sequentially compressing the second flexible tube 14 against the inner wall of the peristaltic pump 18; flowing liquid and air in the second flexible tube 14 to the water reservoir 5 through the second water line 16; and leaking out of the atomization member 9 being prevented because the atmospheric pressure is greater than internal pressure of the essential oil receptacle 8 in which air entering the atomization member 9 and liquid from the first flexible tube 13 flow through the second flexible tube 14 and water flowing through the first flexible tube 13 does not leak out of the atomization member 9. The water flow will slow if the level of water in the water reservoir 5 is low. This is because air enters the atomization member 9. It may take a longer time to draw liquid out of the essential oil receptacle 8. Air in the second flexible tube 14 and the second water line 16 is almost completely drawn out. The liquid may be quickly drawn out if there is liquid in the atomization member 9. The activated the atomization member 9 may atomize liquid flowing from the essential oil receptacle 8. Finally, the atomized particles quickly come out of the nozzle 3.

The second method involves the steps of electrically connecting the controller 24 to the mains electricity; putting the essential oil atomizer on a table or mounting the essential oil atomizer on a wall; filling the water reservoir 5 with water through the inlet 6; pressing the button 4 to activating the essential oil atomizer; the control 24 activating the electric motor 19 to cause the electric motor 19 to clockwise rotate; clockwise rotating the rotatable disc 21 so that the bearings 23 on the shafts 22 clockwise rotate; the bearings 23 sequentially compressing the second flexible tube 14 against the inner wall of the peristaltic pump 18; flowing water in the water reservoir 5 to the essential oil receptacle 8 through the second water line 16 and the second flexible tube 14; drawing air out of the second water line 16, the second flexible tube 14 and the essential oil receptacle 8 in which liquid in the essential oil receptacle 8 is not drawn out completely so that leaking out of the atomization member 9 can be prevented; then the control 24 activating the electric motor 19 to cause the electric motor 19 to counterclockwise rotate; counterclockwise rotating the rotatable disc 21 so that the bearings 23 on the shafts 22 counterclockwise rotate; the bearings 23 sequentially compressing the second flexible tube 14 against the inner wall of the peristaltic pump 18 in which time for drawing liquid is decreased greatly because air in the second water line 16, the second flexible tube 14 and the essential oil receptacle 8 has been drawn out completely by the clockwise rotation of the electric motor 19. As such, liquid from the essential oil receptacle 8 is prevented from leaking out of the atomization member 9. The activated by the control 24 may atomize liquid flowing from the essential oil receptacle 8. Finally, the atomized particles quickly come out of the nozzle 3.

While the invention has been described in terms of preferred embodiments, those skilled in the art will recognize that the invention can be practiced with modifications within the spirit and scope of the appended claims.

What is claimed is:

1. An essential oil atomizer, comprising:
a housing;
an essential oil receptacle disposed in the housing;
first and second fluid connectors connected to the essential oil receptacle;
a water reservoir disposed on a bottom of the housing;
first and second water lines connected to the water reservoir;
a first flexible tube interconnected to the first water line and the first fluid connector;
a second flexible tube interconnected to the second water line and the second fluid connector;
a bracket disposed on the water reservoir;
a peristaltic pump disposed on the bracket;
two openings disposed on one side of the peristaltic pump; and
an electric motor disposed on one surface of the peristaltic pump;
wherein the first flexible tube passes through one opening, inside of the peristaltic pump, and the other opening; and the peristaltic pump includes a rotatable disc operatively connected to the electric motor, three equally spaced shafts secured to rotatable disc, and three bearings put on the shafts respectively.

2. The essential oil atomizer of claim 1, wherein the first fluid connector is below the second fluid connector; both the first and second water lines are inserted into the water reservoir; and ends of both the first and second water lines are adjacent to the water reservoir.

3. The essential oil atomizer of claim 1, wherein the electric motor is secured to a center of the rotatable disc; and the second flexible tube passes through a space between an inner surface of the peristaltic pump and the bearings.

4. The essential oil atomizer of claim, further comprising a slit disposed on the housing and aligned with the water reservoir, a cylindrical member projecting out of the housing, and a nozzle disposed at an end of the cylindrical member.

5. The essential oil atomizer of claim 4, further comprising an inlet disposed on a top of the water reservoir and a sealing cap disposed on the inlet; and wherein the inlet and the sealing cap are threadedly secured together.

6. The essential oil atomizer of claim 5, further comprising an atomization member disposed at an end of the essential oil receptacle, a sealing cap disposed at the other end of the essential oil receptacle and threadedly secured thereto; and wherein the atomization member is aligned with the nozzle.

7. The essential oil atomizer of claim 6, further comprising a plurality of buttons disposed on the end of the cylindrical member adjacent to the nozzle, and a controller disposed in the cylindrical member and electrically connected to the buttons, the atomization member, and the electric motor; and wherein the controller is externally powered.
